(19) United States
(12) Patent Application Publication
(10) Pub. No.: US 2012/0116235 A1
(43) Pub. Date: May 10, 2012

(54) SYSTEMS AND METHODS FOR PRODUCING AUDIBLE INDICATORS THAT ARE REPRESENTATIVE OF MEASURED BLOOD PRESSURE

(75) Inventors: David Trumble, Boulder, CO (US); Peter Galen, Portland, OR (US); Paul Stanley Addison, Edinburgh (GB); James N. Watson, Dunfermline (GB)

(73) Assignee: Nellcor Puritan Bennett LLC, Boulder, CO (US)

(21) Appl. No.: 12/940,796

(22) Filed: Nov. 5, 2010

(51) Int. Cl. A61B 5/021

(52) U.S. Cl. 600/485

(57) ABSTRACT

Systems and methods are disclosed for producing audible indicators that are based on a subject's measured blood pressure. Audible properties of the indicators are processed to represent blood pressure. For example, the duration or volume of the audible indicators may be varied based on the values of the subject's blood pressure. The audible indicators may further be varied based on the subject's blood pressure's deviation from a normal blood pressure and/or previously calculated blood pressure. For example, the audible indicators may be indicative of changes in the subject's blood pressure over time. The audible indicators representing blood pressure may be synchronized with other audible indicators that represent other physiological parameters of the subject, such as, the subject's heart rate.

### SUMMARY OF THE DISCLOSURE

The present disclosure relates to audible indicators that are representative of a subject's blood pressure.

In an embodiment, the subject's blood pressure is measured. An audible indicator that includes one or more audible properties based on the measured blood pressure is produced. In one suitable approach, the duration of an audible indicator may be varied depending on the value of the measured blood pressure. For example, audible indicators with longer durations may be utilized to represent a higher blood pressure and audible indicators with shorter durations may be utilized to represent a lower blood pressure.

In an embodiment, properties of the audible indicators are based on the difference between a normal blood pressure (e.g., normal blood pressure for the subject's demographic) and the measured blood pressure. For example, the pitch of the audible indicator may be higher when the measured blood pressure is above the normal blood pressure, or lower when the measured blood pressure is below the normal blood pressure.

In an embodiment, the properties of the audible indicators are based on the difference between a current blood pressure measurement and a previously determined blood pressure. Thus, audible properties of the audible indicators may represent changes in the subject's blood pressure over time.

In an embodiment, the audible indicators that are representative of the subject's blood pressure may be synchronized based on other physiological parameters of the subject. For example, the audible indicators may be produced synchronously with the subject's pulse rate. In an embodiment, audible indicators that are representative of the subject's other physiological parameters may be modified to additionally represent the subject's blood pressure. For example, audible properties of audible indicators representative of the subject's pulse may be modified based on values of the subject's blood pressure.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with an embodiment;

FIG. 2 is a block diagram of a portion of the illustrative patient monitoring system of FIG. 1 in accordance with an embodiment;

FIG. 3 is a flow chart of illustrative steps performed to produce a waveform that is representative of a patient's blood pressure in accordance with an embodiment;

FIG. 4 is a flow chart of illustrative steps performed to produce a waveform that is representative of a patient's blood pressure and is synchronized with the patient's heart rate in accordance with an embodiment;

FIG. 5 is a flow chart of illustrative steps performed to produce a waveform that is representative of the difference between a patient's blood pressure and a normal blood pressure in accordance with an embodiment;

FIG. 6 is a flow chart of illustrative steps performed to produce a waveform that is representative of changes in a patient's blood pressure in accordance with an embodiment; and

FIG. 7 is a flow chart of illustrative steps performed to modify audible indicators that are representative of a subject's pulse based on the subject's blood pressure in accordance with an embodiment.

### DETAILED DESCRIPTION OF THE DISCLOSURE

To provide an overall understanding of the disclosure, certain illustrative embodiments will now be described, including systems and methods for producing audible indicators of blood pressure. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope hereof.

Monitoring the physiological state of a subject, for example, by determining, estimating, and/or tracking one or more physiological parameters of the subject, may be of interest in a wide variety of medical and non-medical applications. Indications of a subject's physiological parameters obtained from sensors (e.g., temperature sensors, blood pressure cuffs, continuous non-invasive blood pressure sensors, pulse oximeter sensors, regional oxygen saturation sensors, EEG sensors, EMG sensors, EKG sensors, spirometer sensors, and/or any other suitable sensor can provide short-term and long-term benefits to the subject, such as early detection and/or warning of potentially harmful conditions, diagnosis and treatment of illnesses, and/or guidance for preventative medicine. Medical sensors for monitoring multiple parameters are typically connected to one or more devices (e.g., single parameter and multi parameter monitors).

One type of device that can be used to monitor the physiological state of a subject is an oximeter. An oximeter is a medical device that may determine, for example, the oxygen saturation of blood. An oximeter may include a light sensor that is placed at a site on a patient, typically a fingertip, toe, forehead or earlobe, or, in the case of a neonate, across a foot. Herein, a patient may refer to any suitable subject that is being monitored. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured and other physiological parameters such as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less red light and more infrared light than blood with a lower oxygen saturation.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques.

When the measured blood parameter is the oxygen saturation of hemoglobin, a convenient starting point assumes a saturation calculation based on Lambert-Beer's law. The following notation will be used herein:

I(λ,t)=I_{O}(λ)exp(−sβ_{O}(λ)+(1−s)βᵣ(λ))l(t)) (1)

where:
λ=wavelength;
t=time;
I=intensity of light detected;
Iₒ=intensity of light transmitted;
s=oxygen saturation;
βₒ,βᵣ=empirically derived absorption coefficients; and
l(t)=a combination of concentration and path length from emitter to detector as a function of time.

One common type of oximeter is a pulse oximeter, which may indirectly measure the oxygen saturation of a patient's blood (as opposed to measuring oxygen saturation directly by analyzing a blood sample taken from the patient) and changes in blood volume in the skin. In pulse oximetry, by comparing the intensities of two wavelengths at different points in the pulse cycle, it is possible to estimate the blood oxygen saturation of hemoglobin in arterial blood. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

For example, using a pulse oximeter, saturation may be calculated by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o } + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{IR} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{IR} ) - {β}_{r} ( {λ}_{IR} ) )\end{matrix}} \\ Note in discrete time \\ \frac{d log I ( λ , t )}{d t} ≃ log I ( λ , {t}_{2} ) - log I ( λ , {t}_{1} ) \\ Using log A \text{-} log B = log A / B , \\ \frac{d log I ( λ , t )}{d t} ≃ log ( \frac{I ( {t}_{2} , λ )}{I ( {t}_{1} , λ )} ) \\ So , ( 4 ) can be rewritten as \\ \frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) - {β}_{r} ( {λ}_{R} )} .$$

From (5), R can be calculated using two points (e.g., PPG maximum and minimum), or a family of points. One method using a family of points uses a modified version of (5). Using the relationship

$$\begin{matrix}\frac{d log I}{d t} = \frac{d I / d t}{I} \\ now ( 5 ) becomes & ( 6 ) \\ \begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} & ( 7 )\end{matrix}$$

which defines a cluster of points whose slope of y versus x will give R where

x(t)=[I(t₂,λ_{IR})−I(t₁,λ_{IR}]I(t₁,λ_{R})

y(t)=[I(t₂,λ_{R})−I(t₁,λ_{R})]I(t₁,λ_{IR})

y(t)=Rx(t) (8)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

The foregoing is merely illustrative and any suitable processing techniques may be used to calculate pulse oximetry values. For example, Fourier transforms and continuous wavelet transforms may be used to process the PPG signals and derive blood oxygen saturation.

Another common type of oximeter is a regional oximeter, which may be used to calculate an oxygen saturation of a patient's blood in a non-invasive manner. In regional oximetry, by comparing the intensities of two wavelengths of light, it is possible to estimate the blood oxygen saturation of hemoglobin in a region of a body. Whereas pulse oximetry measures blood oxygen based on changes in the volume of blood due to pulsing tissue (e.g., arteries), regional oximetry may examine blood oxygen saturation within the venous, arterial, and capillary systems within a region of a patient. For example, a regional oximeter may include a sensor to be placed on a patient's forehead and may be used to calculate the oxygen saturation of a patient's blood within the venous, arterial and capillary systems of a region underlying the patient's forehead (e.g., in the cerebral cortex). The sensor may include two emitters and two detectors: one detector that is relatively “close” to the two emitters and another detector that is relatively “far” from the two emitters.

For example, if I_{A} represents the intensity of the received/detected light associated with the “close” detector,

$$\frac{{I}_{A} ( λ , t )}{{I}_{O} ( λ )} ,$$

may be derived using Lambert-Beer's law, described above. Similarly, if I_{B} represents the intensity of the received/detected light associated with the “far” detector,

$$\frac{{I}_{B} ( λ , t )}{{I}_{O} ( λ )} ,$$

may be derived using Lambert-Beer's law, described above. The two or more signals may be derived for a variety of light wavelengths, λ. The two derived signals may then be subtracted from each other and processed to arrive at a regional saturation value that pertains to the additional tissue through which the light received at the “far” detector passed (tissue in addition to the tissue through which the light received by the “close” detector passed, e.g., the brain tissue), when it was transmitted through a region of a patient (e.g., a patient's cranium). Other methods to calculate regional blood oxygen saturation are well known in the art.

Another type of device that can be used to monitor the physiological state of a subject is a continuous non-invasive blood pressure (CNIBP) device. PPG sensors may be affixed to a subject and allow for the determination of the subject's blood pressure, for example, using CNIBP monitoring techniques. For example, some CNIBP monitoring techniques have been developed that involve the use of two probes or sensors positioned at two different locations on a subject's body. The elapsed time, T, between the arrival of corresponding points of a pulse signal at the two locations may then be determined using the two probes or sensors. The estimated blood pressure, p, may then be related to the elapsed time, T, by

p=a+b·ln(T) (9)

where a and b are constants that are dependent upon the nature of the subject and the signal detecting devices. Other blood pressure equations using elapsed time may also be used.

Such a CNIBP monitoring technique is described in Chen et al. U.S. Pat. No. 6,566,251, which is hereby incorporated by reference herein in its entirety. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a patient's body. In some approaches, a single sensor or probe location may be used to determine blood pressure, as described in U.S. patent application Ser. No. 12/242,238, filed Sep. 30, 2008, which is hereby incorporated by reference herein in its entirety.

Similar sensors or probes may also be used to detect and/or determine pulses or heartbeats. For example, as described in more detail in U.S. patent application Ser. No. 12/242/908, filed Sep. 30, 2008, which is incorporated by reference herein in its entirety, local minima and maxima points may be identified in a PPG signal. Each minimum may be paired with an adjacent maximum to form an upstroke segment.

Similar sensors or probes may also be used to determine respiration rate and other respiratory properties (e.g., respiratory effort). For example, as described in more detail in U.S. Patent App. Pub. No. 2006/0258921, which is incorporated by reference herein in its entirety, the act of breathing, may cause a breathing band to become present in a scalogram derived from a continuous wavelet transform of a PPG signal. This breathing band may occur at or about the scale having a characteristic frequency that corresponds to the breathing frequency. Furthermore, the features within this band (e.g., the energy, amplitude, phase, or modulation) or the features within other bands of the scalogram may result from changes in breathing rate (or breathing effort) and therefore may be based on various respiratory parameters of a patient.

Other devices and sensors may also be used to determine physiological parameters of a subject. For example, an electrical physiological signal (EPS) sensor may be used to determine such signals as electroencephalographic (EEG) signals, electrocardiography (ECG or EKG) signals, electromyography (EMG) signals, or any other electrical physiological signal. As a further example, magnetic resonance imaging (MRI) may be used to determine physiological parameters. Sensors may also be used to determine a subject's body temperature, a pulse transit times (PTT), or both. In an embodiment, PTT may be determined by using plethysmograph data in conjunction with ECG data. For example, PTT may be determined by comparing an ECG onset point with a PPG arrival point. An ECG signal may be processed in order to detect the QRS complex and to detect the R wave peak. The plethysmograph signal may be processed to detect the pulse timing. The PTT may then be calculated as the time between the R wave peak and the corresponding pulse peak. Other suitable techniques for calculating PTT are well know in the art and may also be used.

A spirometer is another device that measures a physiological parameter of a subject. A spirometer measures the volume of air inspired and expired by a subject's lungs using one or more sensors. There are different types of sensors that can be used for spirometry, including, for example, pressure sensors and wind turbines.

These and other devices and sensors can be used for monitoring physiological parameters of a subject. These devices may be standalone devices or may be combined into one or more multi-parameter monitoring devices. The monitoring devices may be physically connected to each sensor to receive signals from the sensors and perform various processing of the signals. The physiological parameters may, for example, be consolidated and displayed on a single display to provide a condensed view of the subject's physiological state to assist a medical professional in treating the subject. Alternatively, a subject's physiological parameters obtained from the sensor signals may be displayed on multiple displays that may be associated with one or more single parameter and multiple parameter monitoring devices.

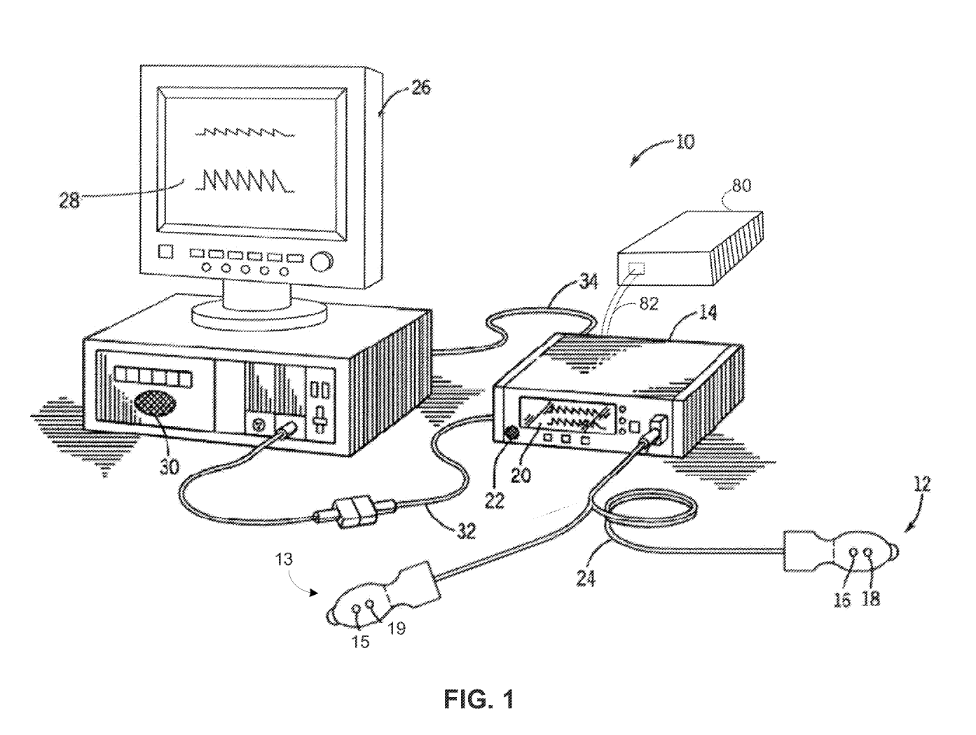

FIG. 1 shows an illustrative patient monitoring system 10, System 10 may include a sensor unit 12 and a monitor 14. In an embodiment, sensor unit 12 is part of a continuous, non-invasive blood pressure (CNIBP) monitoring system. In an embodiment, sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. In an embodiment, system 10 may include one or more additional sensor units, such as sensor unit 13, which may take the form of any of the embodiments described herein with reference to sensor unit 12. For example, sensor unit 13 may include emitter 15 and detector 19. Sensor unit 13 may be the same type of sensor unit as sensor unit 12, or sensor unit 13 may be of a different sensor unit type than sensor unit 12. Sensor units 12 and 13 may be capable of being positioned at two different locations on a subject's body; for example, sensor unit 12 may be positioned on a patient's forehead, while sensor unit 13 may be positioned at a patient's fingertip. As discussed in additional detail below, one or more signals from one or more sensors and/or sensor units may be used in the measurement assessment techniques described herein.

Sensor units 12 and 13 may each detect any signal that carries information about a patient's physiological state, such as the pulsatile force exerted on the walls of an artery using, for example, oscillometric methods with a piezoelectric transducer. According to another embodiment, system 10 may include a plurality of sensors forming a sensor array in lieu of either or both of sensor units 12 and 13. It will be understood that any type of sensor, including any type of physiological sensor, may be used in one or more of sensor units 12 and 13 in accordance with the systems and techniques disclosed herein. It is understood that any number of sensors measuring any number of physiological signals may be used to assess patient status in accordance with the techniques described herein.

According to an embodiment, system 10 may include a plurality of sensors forming a sensor array in lieu of a single sensor, for example, sensor unit 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

In an embodiment, the signal obtained from a sensor or probe, such as sensor units 12 or 13, may take the form of a PPG signal obtained, for example, from a CNIBP monitoring system or pulse oximeter. In this embodiment, sensor units 12 and 13 may each include a light sensor that is placed at a site on a patient, typically a fingertip, toe, forehead or earlobe, or in the case of a neonate, across a foot. The system may pass light using a light source through blood perfused tissue and photoelectrically sense the absorption of light in the tissue. For example, the system may measure the intensity of light that is received at the light sensor as a function of time. The light intensity or the amount of light absorbed may then be used to calculate physiological measurements (e.g., blood pressure and blood oxygen saturation). Techniques for obtaining blood pressure measurements from data are described in more detail in co-pending, commonly assigned U.S. patent application Ser. No. 12/242,867, filed Sep. 30, 2008, entitled “SYSTEMS AND METHODS FOR NON-INVASIVE CONTINUOUS BLOOD PRESSURE DETERMINATION” and co-pending, commonly assigned U.S. patent application Ser. No. 12/242,238, filed Sep. 30, 2008, entitled “SYSTEMS AND METHODS FOR NON-INVASIVE BLOOD PRESSURE MONITORING,” which are both hereby incorporated by reference herein in their entireties.

It will be understood that the present disclosure is applicable to any suitable signals that communicate information about an underlying physiological process. It will be understood that the signals may be digital or analog. Moreover, it will be understood that the present disclosure has wide applicability to signals including, but not limited to other biosignals (e.g., electrocardiogram, electroencephalogram, electrogastrogram, phonocardiogram, electromyogram, pathological sounds, ultrasound, or any other suitable biosignal), or any combination thereof. For example, the techniques of the present disclosure could be applied to monitoring pathological sounds or arterial (or venous) pressure fluctuations.

In an embodiment, sensor units 12 and 13 may be connected to and draw power from monitor 14 as shown. In another embodiment, sensor units 12 and 13 may be wirelessly connected to monitor 14 and include their own batteries or similar power supplies (not shown). In an embodiment, sensor units 12 and 13 may be communicatively coupled to monitor 14 via cables such as cable 24. However, in other embodiments, a wireless transmission device (not shown) or the like may be used instead of or in addition to cable 24.

Monitor 14 may be configured to calculate physiological parameters (e.g., heart rate, blood pressure, blood oxygen saturation) based at least in part on data received from one or more sensor units such as sensor units 12 and 13. In an alternative embodiment, the calculations may be performed on the monitoring device itself and the result of the calculations may be passed to monitor 14. Further, monitor 14 may include a display 20 configured to display the physiological parameters or other information about the system. In the embodiment shown, monitor 14 may also include a speaker 22 and/or speaker 30 to provide an audible sound that may be used in various other embodiments, for example, sounding an audible alarm in the event that a patient's physiological parameters are not within a predefined normal range. In an embodiment, speaker 22 and speaker 30 are substantially similar. Monitor 14 may also include a measurement quality indicator, such as a graphic or text in display 20 or a tone or message via speaker 22. Speaker 22 may also be used to provide various continuous or discontinuous tones related to blood pressure status. Such embodiments are described in greater detail below.

In the illustrated embodiment, system 10 may also include a multi-parameter patient monitor 26. The monitor 26 may include a cathode ray tube display, a flat panel display (as shown) such as a liquid crystal display (LCD) or a plasma display, or may be any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood pressure from monitor 14, blood oxygen saturation generated by monitor 14 (referred to as an “SpO₂” measurement), and pulse rate information from monitor 14. Monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14 via a cable 82, a battery, or by a conventional power source such as a wall outlet, may include any suitable physiological signal calibration device. Calibration device 80 may be communicatively coupled to monitor 14 via cable 82, and/or may communicate wirelessly (not shown). For example, calibration device 80 may take the form of any invasive or non-invasive physiological monitoring or measuring system used to generate reference physiological measurements for use in calibrating a monitoring device. For example, calibration device 80 may take the form of a blood pressure monitoring system, and may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In an embodiment, calibration device 80 may include a manual input device (not shown) used by an operator to manually input reference physiological measurements obtained from some other source (e.g., an external invasive or non-invasive physiological measurement system).

Calibration device 80 may also access reference measurements stored in memory (e.g., RAM, ROM, or a storage device). As described in more detail below, the reference measurements generated or accessed by calibration device 80 may be updated in real-time, resulting in a continuous source of reference measurements for use in continuous or periodic calibration. Alternatively, reference measurements generated or accessed by calibration device 80 may be updated periodically, and calibration may be performed on the same periodic cycle. In the depicted embodiments, calibration device 80 is connected to monitor 14 via cable 82. In other embodiments, calibration device 80 may be a stand-alone device that may be in wireless communication with monitor 14. Reference measurements may then be wirelessly transmitted to monitor 14 for use in calibration. In still other embodiments, calibration device 80 is completely integrated within monitor 14. For example, in an embodiment, calibration device 80 may access reference measurements from a relational database stored within calibration device 80, monitor 14, or multi-parameter patient monitor 26. As described in additional detail below, calibration device 80 may be responsive to a recalibration signal, which may initiate the calibration of monitor 14 or may communicate recalibration information to calibration device 80 (e.g., a recalibration schedule). Calibration may be performed at any suitable time (e.g., once initially after monitoring begins) or on any suitable schedule (e.g., a periodic or event-driven schedule). In an embodiment, calibration may be initiated or delayed based at least in part on a measurement quality assessment or a recalibration initiation assessment. Techniques for recalibrating a continuous, non-invasive blood pressure (CNIBP) system are described in more detail in co-pending, commonly assigned U.S. patent application Ser. No. 12/242,858, filed Sep. 30, 2008, entitled “SYSTEMS AND METHODS FOR RECALIBRATING A NON-INVASIVE BLOOD PRESSURE MONITOR,” which is hereby incorporated by reference herein in its entirety.

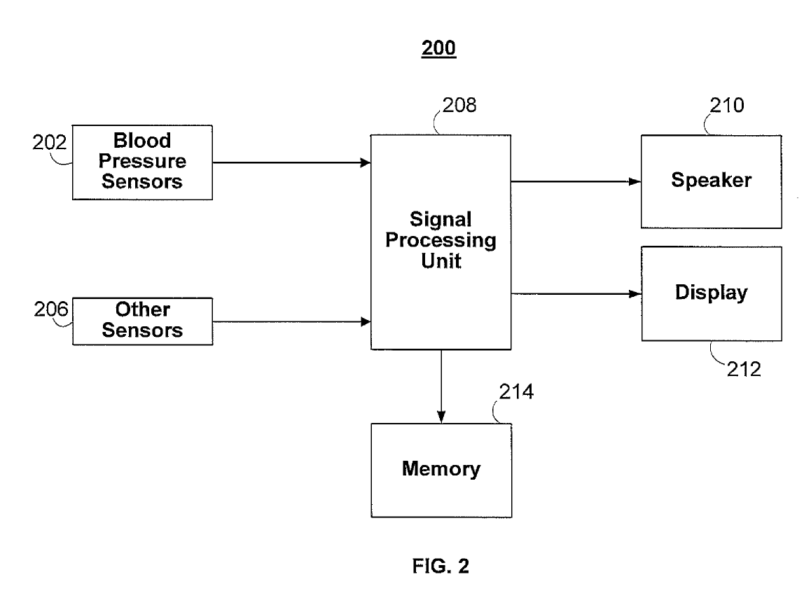

FIG. 2 is block diagram 200, which depicts a portion of physiological monitoring system 10 of FIG. 1. Diagram 200 includes blood pressure sensors 202, other sensors 206, signal processing unit 208, speaker 210, display 212, and memory 214.

Blood pressure sensors 202 may receive one or more signals from which a patient's blood pressure may be determined. The signals may be raw signals representative of any suitable measurement of blood pressure, for example, systolic pressure, diastolic pressure, mean arterial pressure (MAP), or any other suitable type of blood pressure measurement representation. Blood pressure sensors 202 may receive signals using any suitable method, for example, palpation methods, auscultatory methods, oscillometric methods, the methods described above in relation to PPG signals, or any suitable method described above, or any other suitable method. Blood pressure sensors 202 may generate continuous or discontinuous signals. Blood pressure sensors 202 are preferably non-invasive sensors, however, they may be invasive as well. Blood pressure sensors 202 may be implemented in any suitable manner, for example, using one or more PPG signals from a pulse oximeter, a sphygmomanometer, any other implementation described above, or any other suitable implementation. Data acquired by blood pressure sensors 202 is passed to signal processing unit 208 in any suitable manner. In an embodiment, blood pressure sensors 202 are substantially similar to sensor unit 12 and/or sensor unit 13 of FIG. 1.

Other sensors 206 may receive one or more signals from which any other suitable physiological characteristic of a patient may be determined. The signals may include data related to any suitable physiological characteristic, for example, heart rate, pulse, oxygen saturation, respiration rate, breathing effort, body temperature, or any other characteristic described above, or any other suitable characteristic. Other sensors 206 may be implemented in any suitable manner using any suitable device, for example, using the devices describe above. Data acquired by other sensors 206 is passed to signal processing unit 208 in any suitable manner. In an embodiment, other sensors 206 are substantially similar to sensor unit 12 and/or sensor unit 13 of FIG. 1.

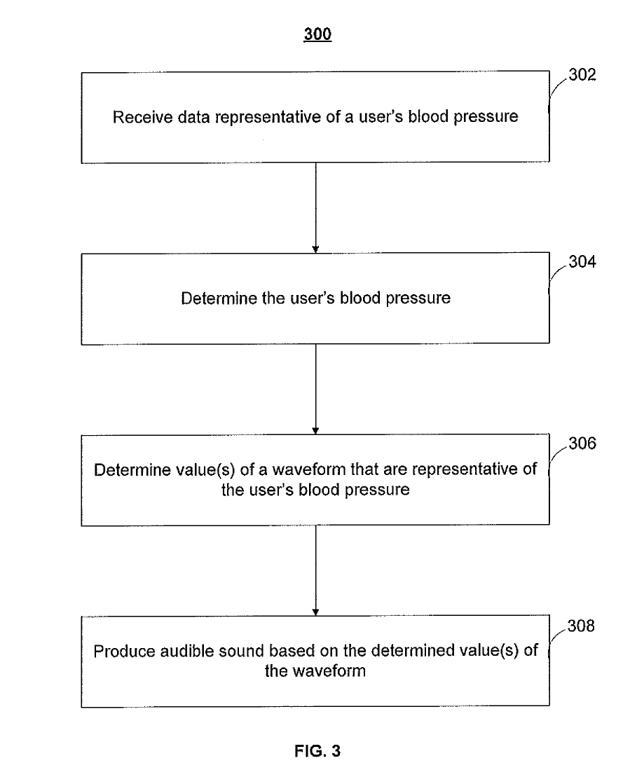

Signal processing unit 208 is generally responsible for receiving, analyzing, and processing data acquired by blood pressure sensors 202 and other sensors 206. For example, signal processing unit 208 may be capable of determining values of the systolic pressure, diastolic pressure, and/or MAP of a patient based on the data received from blood pressure sensors 202. In an embodiment, signal processing unit 208 may be capable of determining changes over time of various aspects of a patient's blood pressure. For example, signal processing unit 208 may be capable of determining how quickly a patient's blood pressure drops and by what magnitude. In an embodiment, signal processing unit 208 provides the determined information to display 212 for visual display to clinicians. In an embodiment, signal processing unit 208 uses the determined information to generate signals for use by speaker 210, for example, electrical signals representative of auditory tones. In an embodiment, properties of the signals are based on the values of the determined blood pressure information. For example, signal processing unit 208 may cause an auditory tone to be modulated, wherein the depth of modulation is based on the magnitude of the determined MAP value. The generation and modulation of auditory tones based on blood pressure are described in further detail below with regard to FIGS. 3-7.

Herein the terms audible indicator, tone, beep, and buzz may represent any suitable waveform and/or sound. Generally, a beep is considered to be a sound or waveform with a relatively short duration, a buzz is a sound or waveform with a longer duration and may have a high pitch, and a tone is a sound or waveform with a longer duration than a buzz. However, any specific reference to a type of sound or waveform is done for illustrative purposes. Each of the tone, beep, buzz, or any other suitable audible indicator and/or waveform may be equally utilized in place of one another. Herein an audible waveform is considered to be an electrical, magnetic, optical, mathematical, and/or auditory signal and may be infinite or finite. In an embodiment discussed herein, an audible waveform is an electrical, optical, and/or mathematical signal that may be processed by, for example, system 10 to produce an audible sound.

Signal processing unit 208 may be implemented using any suitable hardware and/or software. For example, algorithms for processing blood pressure information from blood pressure sensors 202 to form audible waveforms that may be converted into audible tones by speaker 210 may be implemented on any suitable processor, for example, a digital signal processor manufactured by Texas Instruments. Additionally, or alternatively, signal processing unit 208 may be any suitable analog circuitry to process the received data from sensors 202 and 206 and/or produce an output that is suitable for speaker 210 and/or display 212. For example, any suitable resistor, capacitor, inductor, transistor, amplifier, comparator, filter, or any other suitable electrical component may be utilized to implement signal processing unit 208.

Speaker 210 may be substantially similar to speakers 22 and/or 30 of FIG. 1. In an embodiment, speaker 210 may receive processed data from signal processing unit 208 to, for example, produce audible tones that are representative of a patient's blood pressure status and/or any other suitable physiological parameter. Display 212 may be substantially similar to displays 20 and/or 28 of FIG. 1. Display 212 may receive processed data from signal processing unit 208 to, for example, display visual information representative of a patient's blood pressure status and/or any other suitable physiological parameter.

Memory 214 may be any suitable form of memory. For example, memory 214 may be any suitable form of read-only memory and/or random access memory. Memory 214 is illustrated by way of example, and not limitation. Any suitable computer-readable media may be used in the system for data storage. Computer-readable media are capable of storing information that can be interpreted by, for example, signal processing unit 208. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store desired information. In an embodiment, any suitable component within diagram 200 may read from and/or write to memory 214. For example, in an embodiment, display 212 and speaker 210 may write and read information to and from memory 214.

FIG. 3 shows illustrative process 300 for producing an audible sound that is based on a patient's measured blood pressure. At step 302, data that is representative of a patient's blood pressure is received at, for example, signal processing unit 208 of FIG. 2 from, for example, blood pressure sensors 202. This data may be of any suitable form. For example, the data may be raw data related to light intensity measurements of a PPG attached to one or more locations on a patient. At step 304, the patient's blood pressure is determined. For example, the raw data received at step 302 may be processed into numerical values that represent the patient's blood pressure in, for example, standard blood pressure units such as millimeters of mercury (mmHg). For example, the data received may be processed to form values that represent, for example, diastolic pressure, systolic pressure, and/or MAP, and/or any other suitable blood pressure representation. The processing of the data received at step 302 to the values representative of blood pressure may be performed using any suitable method, for example, using the methods described above. In an embodiment, the data received at step 302 are numerical values that represent the patient's blood pressure in standard blood pressure units. In such embodiments, further processing of the data received at step 302 may not be necessary at step 304. For example, the data received from blood pressure sensors 202 may already be in a suitable blood pressure representation.

At step 306, one or more values of a waveform that is representative of the patient's blood pressure are determined by, for example, signal processing unit 208 of FIG. 2. The values representative of the sound may be determined and/or created in any suitable manner. For example, signal processing unit 208 may calculate waveform values representative of the sound. The waveform may be a discontinuous waveform, for example, a digitally sampled waveform. Additionally, or alternatively, the waveform may be a continuous waveform, for example, an analog signal.

The waveform may be based on a mathematical formula representative of any suitable waveform. For example, the waveform may have any suitable magnitude, amplitude, and/or frequency. The waveform may have any suitable property, such as being continuous, discontinuous, periodic, non-periodic, sinusoidal, square, triangle, saw tooth, impulse train, linear, non-linear, modulated, and/or any other suitable waveform property. For example, the waveform may be a wave comprised of non-zero sample values and zero sample values, wherein the number of non-zero samples are less than, greater than, or equal to the number of zero samples. For example, when this waveform is passed to an audio transducer, such as speaker 210 of FIG. 2, a beep sound would be produced, wherein the duration of the beep sound would be related to the number of non-zero samples. As another example, the waveform may be a sinusoidal waveform with a particular frequency. Such a waveform would produce a monotone at the particular frequency when passed to an audio transducer. In an embodiment, the waveform may be representative of speech sounds. For example, the waveform may be representative of a person stating numbers, alerts, and/or any other suitable information.

In an embodiment, the determination of the values representative of the sound may be calculated in real-time, near real-time, and/or be predetermined. For example, signal processing unit 208 may continuously solve the mathematical formula to produce new sound sample values. As another example, signal processing unit 208 can continuously produce a continuous waveform using analog circuitry. Alternatively, or additionally, sound sample values may be predetermined and stored in any suitable storage device, for example, memory 214 of FIG. 2. In such embodiments, as signal processing unit 208 proceeds through process 300, signal processing unit 208 may retrieve the sound values from the storage device. In an embodiment, the number of sample values produced, how the sample values are produces, and/or when the samples are output from signal processing unit 208 depends on a signal processing sampling rate. The sampling rate may be of any suitable value and may be variable and/or non-variable.

In an embodiment, the values of the waveform are based on the patient's blood pressure. For example, elements of the waveform formula that are used to determine sample values of the sound at step 306 may be changed in a manner that is based on the blood pressure values. For example, the magnitude, period, and/or other properties of the waveform may be adjusted depending on a patient's blood pressure.

For example, if the waveform is the waveform described above with zero and non-zero sample values, the duration of the zero and non-zero samples may be increased or decreased based on the value of the patient's blood pressure. For example, the number of non-zero samples in the waveform may be directly related to the determined value of MAP. For example, if the determined MAP is 100 mmHg, there may be 100 zero samples followed by 100 non-zero samples during a period of time. If the MAP changes to 80 mmHg, there may be 120 zero samples followed by 80 non-zero samples during the same period of time. This would produce a tone that is shorter in duration and more spaced out than the tone produced when MAP is 100 mmHg. A clinician would be able to interpret this change of sound as a change in MAP. In an embodiment, samples of the tone may be processed to represent changes in blood pressure such that the number of zero value samples remains constant, but the number of non-zero value samples changes (with a constant sample rate). This would produce tones that vary in duration, but are equally spaced apart.

Alternatively, samples of the tone may be processed to represent changes in blood pressure such that number of non-zero value samples remain constant, but the number of zero value samples changes (with a constant sample rate). This would produce tones that are equal in duration, but vary in spacing. In an embodiment, similar changes in tone duration may be achieved by varying the sample rate according to the value of blood pressure. The duration of the tone may be changed such that the duration has a linear relationship to the value of a patient's blood pressure. Alternatively, the duration of the tone may be changed such that the duration has a non-linear relationship to the value of a patient's blood pressure. For example, the duration of tones and spaces between tones may become exponentially smaller as blood pressure rises. Such an embodiment would produce a rapid tone rate at high blood pressures and slower tones at low blood pressures. Any suitable change to the duration of a tone, spaces between tones, and/or correlation to a patient's blood pressure may be utilized without departing from the scope of this disclosure.

When the waveform is a continuous waveform, for example an analog signal, the duration of the tone and/or spaces between tones may be implemented using, for example, any suitable circuitry. For example, waveform generation circuitry with a switch connected to ground. For example, the switch may be closed to change any non-zero values in a signal to zero values. Alternatively, the switch may be opened to have no effect on the waveform. The amount of time the switch remains opened or closed may be based on a patient's blood pressure as discussed above with regard to changing the number of zero value or non-zero value samples. The switch may be implemented in any suitable manner. Creating this change in a continuous waveform is not limited to the use of a switch, but may be implemented in any suitable manner.

In an embodiment, the volume and/or pitch of the tone may vary based on the value of blood pressure. For example, the amplitude of the waveform may be based on the value of a patient's blood pressure. Varying the amplitude of the waveform changes the volume of the sound when output to, for example, speaker 210 of FIG. 2. For example, if a patient's MAP is 100 mmHg, the amplitude of the waveform can be adjusted to 100. If the patient's MAP increases to 120 mmHg, the amplitude of the waveform can be adjusted to 120. A clinician would be able to interpret the increase in volume of the sound as an increase in MAP. As another example, if the patient's MAP decreases to 80 mmHg, the amplitude of the waveform can be adjusted to 80. A clinician would interpret the decrease in volume of the sound as a decrease in MAP.

As another example, the frequency of the sound waveform may be based on the value of a patient's blood pressure. Varying the frequency of the waveform changes the pitch of the sound when output. For example, if a patient's MAP is 100 mmHg, the frequency of the waveform can be adjusted to 100 Hz. If the patient's MAP increases to 120 mmHg, the frequency of the waveform can be adjusted to 120 Hz. A clinician would be able to interpret the increased pitch of the sound as an increase in MAP. As another example, if the patient's MAP decreases to 80 mmHg, the frequency of the waveform can be adjusted to 80 Hz. A clinician would interpret the decreased pitch of the sound as a decrease in MAP.

As another example, the waveform may be a modulated signal, for example, analog modulated (e.g., amplitude modulated, frequency modulated, phase modulated), and/or digitally modulated (e.g., phase-shift keying, frequency-shift keying, amplitude-shift keying), and/or any out suitable form of modulation. In such embodiments, the waveform's depth of modulation may be based on a patient's blood pressure, for example, how much a signal modulates around its original pre-modulation level may be varied depending on a patient's blood pressure level. For example, if a patient's MAP is 100 mmHg, the depth of modulation of the signal may be 50%. For example, in the case of amplitude modulation, this can have an effect of a tone varying in amplitude from 0.5 to 1.5 in a period of time. If the patient's MAP increases to 120 mmHg, the depth of modulation of the waveform may be increased to 70%. For example, in the case of amplitude modulation, this would cause the volume range of the tone to increase, for example, to an amplitude range of 0 to 2 in the same period of time. A frequency modulated signal would create a tone that has varying pitch. In the case of frequency modulation, the change in depth of modulation would change what frequency ranges the signal sweeps between and how fast the sweep occurs. A clinician would be able to detect the change in frequency sweeps and determine that a change in the patient's blood pressure has occurred.

In an embodiment, the waveform is, or may include, values representing speech sounds. For example, speech sounds may be produced to indicate the patient's blood pressure or change in pressure. For example, if the patient's blood pressure is determined to be 100 mmHg, speech sounds may indicate that the blood pressure is 100 mmHg by stating “100” at an appropriate time. The speech sounds may state any suitable information. For example, the speech sounds may state, “current blood pressure is 100 mmHg.”

In an embodiment, the speech sounds may be included with the beeps and/or tones described above. For example, the speech sounds may be inserted over a beep and/or tone sound, such that a beep and/or tone would be produced during the production of the speech sounds. In an embodiment, the speech sounds may be inserted in between the beeps and/or tones, such that the speech sounds would be produced in between audible beeps and/or tones. In an embodiment, the speech sound may be included in the waveform when the determined blood pressure is at designated levels. For example, the speech sound may be included in the waveform to indicate every 10 mmHg of blood pressure. For example, no speech sound would be present at 95 mmHg, but speech sounds would be present at 100 mmHg.

In an embodiment, timestamps including information regarding when the last speech sound was included in the waveform may be stored in any suitable storage device, for example, memory 214 of FIG. 2. The timestamps may be utilized to determine when the last speech sound was included in the waveform. For example, if a patient's blood pressure changes relatively frequently around a designated level, it may not be desirable to insert a speech sound every time the patient's blood pressure is at the designated level. In such embodiments, the waveform may only include the speech sound after a certain amount of time after the last speech sound was included. For example, the speech sounds may not be included within 5 seconds of each other. In an embodiment, the speech sound delay requirement may be ignored when there are relatively drastic changes in a patient's blood pressure. For example, if a patient's blood pressure is quickly rising or falling, it may be preferable to insert the speech sounds in the waveform more frequently, so that clinicians are aware of the relatively drastic changes. In such embodiments, the speech sounds may include an alert, for example, the speech sound may state, “alert, blood pressure changing quickly.”

In an embodiment, the values of the waveform may be based on a table of values. For example, predetermined values of multiple waveforms may be stored in any suitable location, for example, memory 214 of FIG. 2. Each of the multiple waveforms may be associated with a particular blood pressure value or range of blood pressure values. When a patient's blood pressure is determined, signal processing unit 208 may output the waveform associated with the determined blood pressure. For example, a first waveform that is representative of a soft beep sound at a low pitch may be associated with blood pressure values 70 mmHg to 100 mmHg. A second waveform that is representative of a loud beep sound at a high pitch may be associated with blood pressures above 100 mmHg. When a patient's blood pressure is measured to be in between 70 and 100 mmHg, the first waveform will be output. When the patient's blood pressure is measured to be over 100 mmHg, the second waveform will be output.

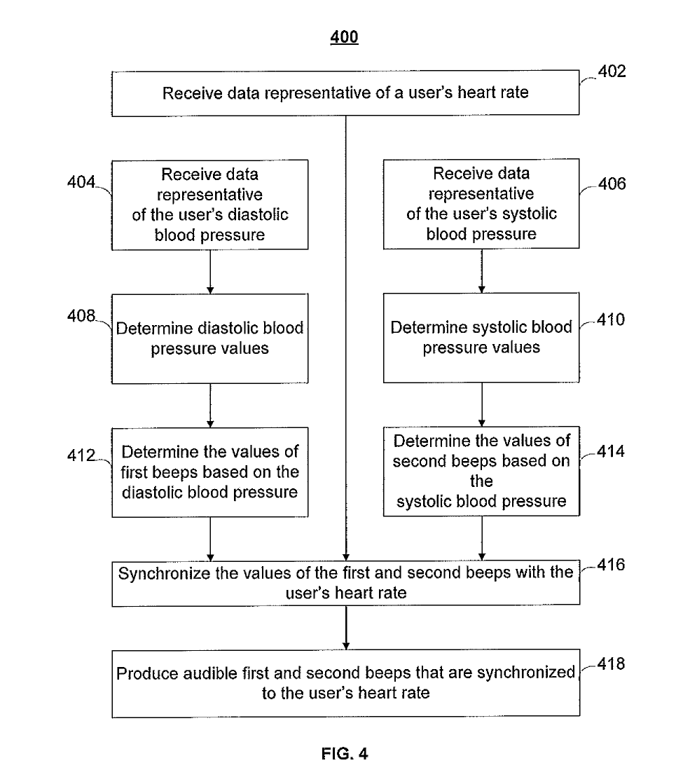

It should be noted that the methods disclosed above for producing and processing waveforms are presented for purely illustrative purposes and are not meant to be limiting as any suitable audible indicator and/or waveform processing that is based on a patient's blood pressure is within the scope of this disclosure. For example, in the above examples, changes of a patient's MAP were used to produce changes in the tone; however, similar changes to the tone may be implemented according to changes in any other suitable form of blood pressure measurement. As another example, all changes may be linear or non-linear in nature. The changes may be directly related or inversely related to changes in blood pressure. For example, when a patient's blood pressure increases, the depth of modulation of a waveform may be decreased. In an embodiment, multiple tone processing methods may be utilized to indicate a patient's blood pressure. For example, both the frequency and amplitude of the waveform may be varied based on the patient's blood pressure. Further examples of modification to the waveform are discussed in greater detail below with respective to FIGS. 4-7.

At step 308, the values of the waveform determined at step 306 are output to a transducer to produce an audible sound based on the waveform. For example, the determined waveform may represent electrical amplitudes of a signal over time. The transducer, such as speaker 210 of FIG. 2, converts the electrical signal into a sound audible by humans.

In practice, one or more stages shown in process 300 may be combined with other stages, performed in any suitable order, performed in parallel (e.g., simultaneously or substantially simultaneously), or removed. For example, the determination of blood pressure at step 304 and the determination of values of a waveform at step 306 may occur substantially simultaneously. Process 300 may be implemented using any suitable combination of hardware and/or software in any suitable fashion. It should be noted that the processes described herein refer to determining a value(s) of a waveform for illustrative purposes. Any suitable type of selection and/or processing of a waveform is within the scope of this disclosure.

FIG. 4 shows illustrative process 400 for producing an audible tone that is synchronized with a patient's heart rate and based on a patient's measured systolic and diastolic blood pressure. In an embodiment, the steps of process 400 may be equally applicable to any other physiological parameter instead of, or in addition to, heart rate. For example, process 400 may utilize oxygen saturation level, or any other suitable parameter discussed above, or any other suitable physiological parameter. At step 402, data representative of a patient's heart rate is received. For example, the data may have been produced and/or measured by other sensors 206 of FIG. 2. The data may be received by, for example, signal processing unit 208 of FIG. 2. Signal processing unit 208 may, for example, discern the timing of the patient's heartbeats from the received data. Data representative of the patient's diastolic and systolic blood pressure are received at step 404 and step 406, respectively. The data may be received from, for example, blood pressure sensors 202 of FIG. 2. Diastolic and systolic blood pressure values are determined at step 408 and step 410, respectively. The values may be determined by, for example signal processing unit 208. Note, the data received at steps 404 and 406 may be the same data and the values determined at steps 408 and 410 may be determined from one calculation that determines both diastolic and systolic blood pressure values. For example, signal processing unit 208 may determine the values of diastolic and/or systolic blood pressure as represented in mmHg, or any other suitable representation. In an embodiment, step 408 and/or step 410 may be substantially similar to step 304 of FIG. 3.

At step 412, values of beeps are determined based on the diastolic blood pressure. For example, the beeps may be based on a waveform of short sinusoids with suitable amplitudes that occurs periodically. The values of the amplitudes may be based on the determined diastolic blood pressure. For example, if the determined diastolic blood pressure is 100 mmHg, the amplitude of the peaks of the sinusoid may be 100. If the diastolic blood pressure changes to 120 mmHg, the amplitude of the peaks of the sinusoid may change to 120. Alternatively, or additionally, the spacing between the sinusoids may be based on the determined diastolic blood pressure. For example, when a patient's diastolic blood pressure is 100 mmHg, the spacing between the sinusoids may be 100 milliseconds. If the patient's diastolic blood pressure changes to 120 mmHg, the spacing between the sinusoids may change to 80 milliseconds. The beeps at step 412 may be based on any other suitable waveform such as, for example, those described with respect to process 300 of FIG. 3. The beeps may be determined to represent diastolic blood pressure or changes in diastolic blood pressure in any suitable manner such as, for example, in the manners described above with regard to process 300 of FIG. 3.

At step 414, values of beeps are determined based on the systolic blood pressure. The beeps may be any suitable beep and may be determined in any suitable manner, such as the manner described with respect to step 412.

At step 416, the values of the first and second beeps are synchronized with the patient's heart rate. For example, beep1 represents the beep based on diastolic blood pressure of step 412, beep2 represents the beep based on systolic blood pressure of step 414, and hr represents a tone that is representative of the patient's heartbeats or heart rate. Beep1, beep2, and hr may be synchronized such that beep1 and beep2 occur sequentially before and/or after hr. Alternatively, or additionally, the sequence may be beep1, hr, and then beep2. In an embodiment, one or both of beep1 and beep2 may occur at the same or substantially the same time as hr. The tone representative of the patient's heartbeats or heart rate may be based on any suitable waveform, for example, those discussed above with regard to process 300 of FIG. 3. In an embodiment, the audible indicators that are representative of the patient's blood pressure (e.g., beep1 and/or beep2) may occur at substantially the same time as the subject's pulse. In such an embodiment, an audible indicator that is representative of the subject's heart rate (e.g., hr) may not be produced. In an embodiment, beep1, beep2, and/or hr may be speech sounds that are based on the patient's diastolic blood pressure, systolic blood pressure, or heart rate, respectively. The speech sounds may be implemented as described above with regard to process 300 of FIG. 3.

At step 418, the synchronized first and second beeps, and heart rate tone are output to, for example, speaker 210 of FIG. 2, to produce an audible sequence. A clinician would be able to discern a change in pulse pressure from changes between, for example, beep1 and beep2. For example, if the volume difference or spacing between the two beeps widens or narrows, a clinician would be to discern that there is a change in at least one of the patient's diastolic and systolic blood pressure. In an embodiment, the first and second beeps can be synchronized with any other physiological parameter in addition to, or alternative to, the patient's heart rate.

In practice, one or more stages shown in process 400 may be combined with other stages, performed in any suitable order, performed in parallel (e.g., simultaneously or substantially simultaneously), or removed. For example, the determination of the values of the first beeps at step 412 and the synchronization of the first and second beeps with the patient's heart rate at step 416 may occur substantially simultaneously. Note that process 400 may be modified to include a determination of MAP values and beeps based on the MAP values instead of, or in addition to, the illustrated diastolic and systolic blood pressure determinations. Process 400 may be implemented using any suitable combination of hardware and/or software in any suitable fashion.

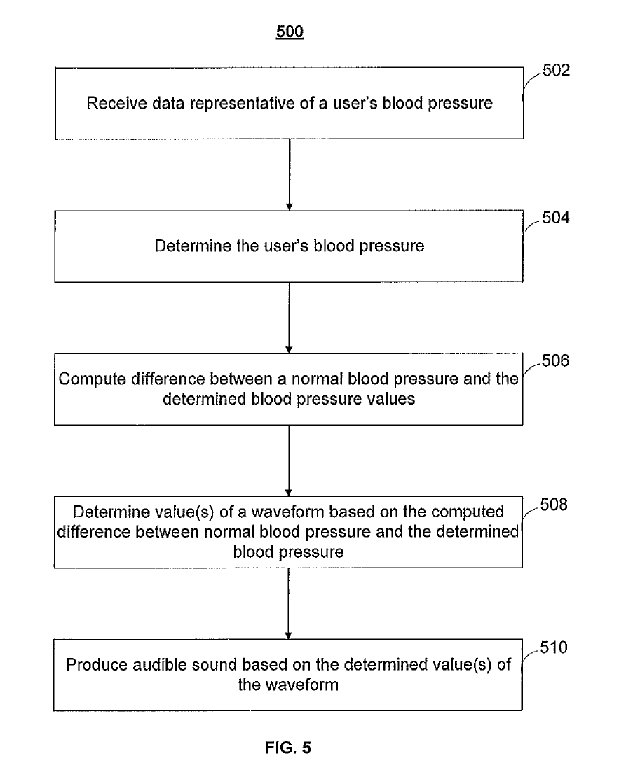

FIG. 5 shows illustrative process 500 for producing an audible tone that is indicative of the difference between a patient's blood pressure and a normal blood pressure. Herein, normal blood pressure may be any suitable blood pressure value or range of values. For example, normal blood pressure may be predetermined and/or based on, for example, clinical studies that determined the normal range of a human's blood pressure. In an embodiment, the normal blood pressure value may be tailored to the individual patient. For example, the normal blood pressure value used may be a normal blood pressure for people of a particular age, sex, and/or any other suitable demographic. In an embodiment, the normal blood pressure value may be based on the patient's medical history. For example, a patient's medical history may indicate that the patient's average resting MAP is 120 mmHg. In an embodiment, the normal blood pressure value used in process 500 may be based on a continuous average. For example, if a patient's blood pressure is monitored over the course of a period of time, the normal blood pressure may be the average blood pressure measured over that period of time. In an embodiment, the normal blood pressure value used in process 500 may be set by a user.

At step 502, data representative of a patient's blood pressure is received. Step 502 may be substantially similar to step 302 of FIG. 3. At step 504, the patient's blood pressure is determined. Step 504 may be substantially similar to step 304 of FIG. 3.

At step 506, a difference between a normal blood pressure and the blood pressure values determined at step 504 is computed. For example, a normal MAP may be 100 mmHg. If the determined MAP at step 504 is 120 mmHg, the difference computed would be 20 mmHg above normal. This computation may be implemented in any suitable manner in, for example, signal processing unit 208.

At step 508, values of a waveform are determined based on the computed difference between normal blood pressure and the determined blood pressure. For example, the waveform may be computed as discussed above with regard to process 300 and process 400 of FIGS. 3 and 4, respectively; however, the waveform would be based on the computed difference as opposed to, or in addition to, the measured blood pressure values themselves. For example, the amplitude of the waveform may be directly based on the difference between normal blood pressure and the determined blood pressure, as opposed to, the determined blood pressure alone.

In an embodiment, the waveform may include speech sounds that are indicative of the computed difference between normal blood pressure and the determined blood pressure. For example, if normal blood pressure is 70 mmHg to 100 mmHg, and the determined blood pressure is 120 mmHg, the speech sounds may state “high” or “above normal” to indicate that the determined blood pressure is above the normal range. Additionally, or alternatively, the speech sounds may state “low” or “below normal” when the determined blood pressure is below normal. The speech sounds may be inserted into the waveform of step 508 as described above with regard to process 300 and process 400 of FIGS. 3 and 4, respectively.

In an embodiment, the waveform may be indicative of whether the determined blood pressure is above or below normal blood pressure. For example, a waveform that linearly or nonlinearly sweeps through multiple values of a particular property or properties of the waveforms. For example, a non-linear frequency sweep may increment from 100 Hz to 500 Hz to 1000 Hz to 10000 Hz over a particular period of time. A waveform that includes a linear or nonlinear frequency sweep from a low frequency to a high frequency over a short period of time may be used to indicate that a patient's blood pressure that is above normal. As an additional example, the waveform can include a linear or nonlinear frequency sweep from a high frequency to a low frequency over a short period of time to indicate that a patient's blood pressure is below normal. In an embodiment, the waveform may include a linear or nonlinear amplitude sweep. For example, an amplitude sweep from a low amplitude to a high amplitude may be used to indicate that a patient's blood pressure is above normal. As an additional example, an amplitude sweep from a high amplitude to a low amplitude can be used to indicate that a patient's blood pressure is below normal. In an embodiment, both a frequency and amplitude sweep may be utilized simultaneously or substantially simultaneously.

Waveforms with such sweeps may have the effect of sounding as if a tone goes up or down in volume or in pitch over the duration of the waveform. A clinician would be able to discern that, for example, a tone that goes up in pitch during its duration indicates that the patient's blood pressure is above normal.

In an embodiment, the range of the sweep, magnitude of change in the sweep, the values in the sweep, the duration of the sweep, or any other suitable parameter of the sweep may be dependent on the magnitude of the difference between the determined blood pressure and a normal blood pressure. For example, a determined blood pressure that is slightly above normal blood pressure may be associated with a waveform that sweeps over low frequencies over a relatively long duration. This may create an effect of a slow, low pitch tone. A determined blood pressure that is significantly above normal blood pressure may be associated with a waveform that sweeps over high frequencies over a relatively short duration. This may create an effect of a fast, high pitch beep, which may attract more attention from a clinician than the slow, low pitch tone.

In an embodiment, the direction of the sweep may be directly or inversely related to whether the determined blood pressure is above or below normal blood pressure. The sweeps may rise or fall in a continuous or discontinuous manner. Any suitable step-size of values in the sweep may be utilized. In an embodiment, a determined blood pressure that is at a normal blood pressure or within a normal blood pressure range may be represented by a waveform that does not include a sweep and/or other waveform modifications. The sweeps may be performed on any suitable waveform, for example, those described in relation to process 300 and/or process 400 of FIGS. 3 and 4, respectively.

In an embodiment, the indication of the patient's blood pressure deviation from normal may be inserted into a waveform that is also indicative of other parameters. For example, the waveform may contain beeps or tones that are indicative of measured blood pressure, heart rate, or any other suitable physiological parameter. The beeps or tones indicative of the physiological parameters may be implemented as discussed above with regard to process 300 and process 400 of FIGS. 3 and 4, respectively.

In an embodiment, a buzz or lack of buzz in spaces between the beeps or tones representing other physiological parameters may be utilized to indicate the difference value between normal blood pressure and the determined blood pressure computed at step 508. For example, a buzz may be sounded as an alarm to indicate that the subject's blood pressure has deviated from normal without obscuring audible indicators that represent other physiological parameters. For example, if the patient's blood pressure is above or below a normal blood pressure, a buzz may be inserted in between spaces of beeps and/or tones that represent, for example, SpO₂ levels and/or MAP value. The beeps and/or tones may represent any suitable physiological parameter. No buzz may be sounded if the patient's blood pressure is at a normal level. The buzz may be based on any suitable waveform, for example, those described above with regard to waveform sweeps or with regard to process 300 and/or process 400 of FIGS. 3 and 4, respectively. For example, the buzz itself may have audible properties that indicate the magnitude of the difference between the determined blood pressure and normal blood pressure in additional to being inserted in between beeps or tones. In a preferred embodiment, the buzz is audibly distinct from the beeps or tones that represent other physiological parameters.

At step 510, an audible sound is produced based on the values of the waveform determined at step 508. Step 510 may be substantially similar to step 308 of FIG. 3.

In practice, one or more stages shown in process 500 may be combined with other stages, performed in any suitable order, performed in parallel (e.g., simultaneously or substantially simultaneously), or removed. For example, the computation of the difference between a normal blood pressure and the determined blood pressure at step 506 and the determination of the values of the waveform at step 508 may occur substantially simultaneously. Process 500 may be implemented using any suitable combination of hardware and/or software in any suitable fashion.

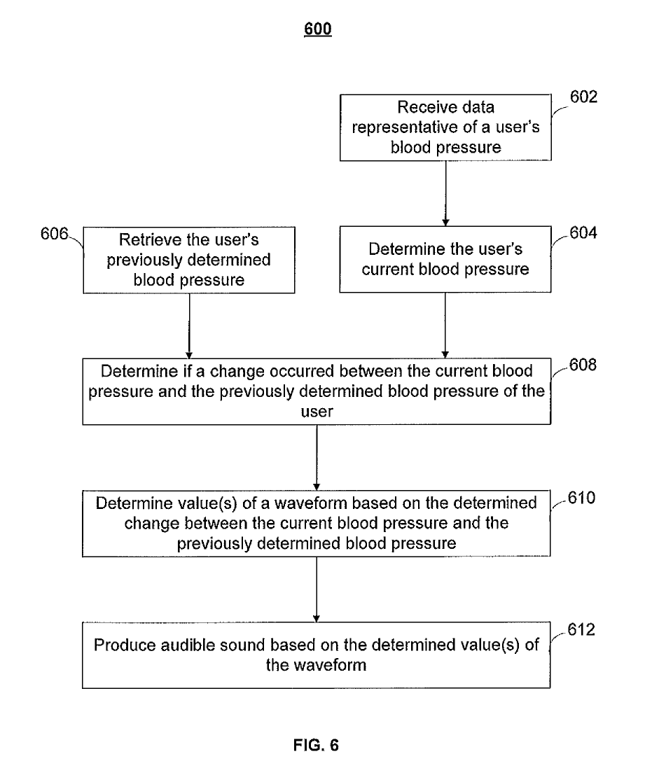

FIG. 6 shows illustrative process 600 for producing an audible tone that is indicative of a change between a patient's current blood pressure and a previously determined blood pressure. At step 602, data representative of a patient's blood pressure is received. Step 602 may be substantially similar to step 502 of FIG. 5. At step 604, the patient's current blood pressure is determined. Step 604 may be substantially similar to step 504 of FIG. 5.

At step 606, a patient's previously determined blood pressure is retrieved from any suitable storage device such as, for example, memory 214 of FIG. 2. In an embodiment, records of past blood pressure measurements and/or other physiological measurements may be stored in the suitable storage device. Any suitable number of past measurements may be stored in the storage device in any suitable form. The previously determined blood pressure may be any suitable blood pressure value or values that were measured in the past. For example, the previously determined blood pressure may be the last measurement of the patient's blood pressure, a blood pressure that was measured several measurements prior, numerous prior blood pressure values, and/or a combination of such measurements.

At step 608, the change between the patient's previously determined blood pressure and the current blood pressure is determined by, for example, signal processing unit 208 of FIG. 2. For example, the change may be computed by subtracting the previous blood pressure value from the current blood pressure. For example, if the previous blood pressure was 100 mmHg, and the current blood pressure is 80 mmHg, it will be determined that the current blood pressure decreased by 20 mmHg. If the previous blood pressure represents multiple past measurements, the average of the past measurements may be utilized to compute the subtraction. In an embodiment, the magnitude of the change in blood pressure may be determined by, for example, computing the absolute value of the change in blood pressure. In an embodiment, the rate of change may additionally, or alternatively, be determined. For example, the slope between the current and the previously determined blood pressure may be determined over time.

At step 610, the value of a waveform is determined based on the determined change between the current blood pressure and the previously determined blood pressure by, for example, signal processing unit 208 of FIG. 2. For example, the waveform may be computed as discussed above with regard to process 300, process 400, and process 500 of FIGS. 3, 4, and 5, respectively; however, the waveform would be based on the determined change between the current blood pressure and previous blood pressure as opposed to, or in addition to, for example, the measured blood pressure values themselves. For example, the amplitude and/or pitch of the waveform may be directly based on the magnitude of change, value of change, and/or rate of change in a patient's blood pressure, as opposed to, the current blood pressure value alone. As another example, the waveform may include speech sounds that indicate the magnitude of change, value of change, and/or rate of change in a patient's blood pressure. For example, if blood pressure is determined to drop by 1 mmHg, the speech sounds may state, “blood pressure is dropping 1 mmHg per second” or provide any other suitable indication of the change in the patient's blood pressure. The speech sounds may be included in the waveform in any suitable manner as described above with regard to process 300, process 400, and/or process 500 of FIGS. 3-5, respectively.

In an embodiment, a waveform with the sweeps described above with regard to process 500 may be utilized to indicate the direction and rate of change. For example, a sweep that includes an increasing pitch (e.g., a sweep from a low frequency to a high frequency) may be utilized to indicate that patient's blood pressure is trending upward. Additionally, a sweep that includes a decreasing pitch (e.g., a sweep from a high frequency to a low frequency) may be utilized to indicate that a patient's blood pressure is trending downward. In an embodiment, the properties of the sweep, for example, the range of the sweep, magnitude of change in the sweep, the values in the sweep, the duration of the sweep, the speed of the sweep, or any other suitable parameter of the sweep, may be based on the change in blood pressure. For example, a blood pressure that has a relatively slow rate of change may be associated with a waveform that sweeps over low frequencies over a relatively long duration. This may create an effect of a slow, low pitch tone. A blood pressure that a relatively fast rate of change may be associated with a waveform that sweeps over high frequencies over a relatively short duration. This may create an effect of a fast, high pitch beep, which may attract more attention from a clinician than the slow, low pitch tone.

Any suitable waveform, or combination of waveforms, may be utilized to represent a patient's change in blood pressure in any suitable manner, such as the methods described in regard to process 300, process 400, and/or process 500 of FIGS. 3, 4, and 5, respectively.

At step 612, an audible sound is produced based on the values of the waveform determined at step 610. Step 612 may be substantially similar to step 512 of FIG. 5.

In practice, one or more stages shown in process 600 may be combined with other stages, performed in any suitable order, performed in parallel (e.g., simultaneously or substantially simultaneously), or removed. For example, the determination of a patient's current blood pressure at step 604 and the retrieval of the patient's previously determined blood pressure at step 606 may occur substantially simultaneously. Process 600 may be implemented using any suitable combination of hardware and/or software in any suitable fashion.

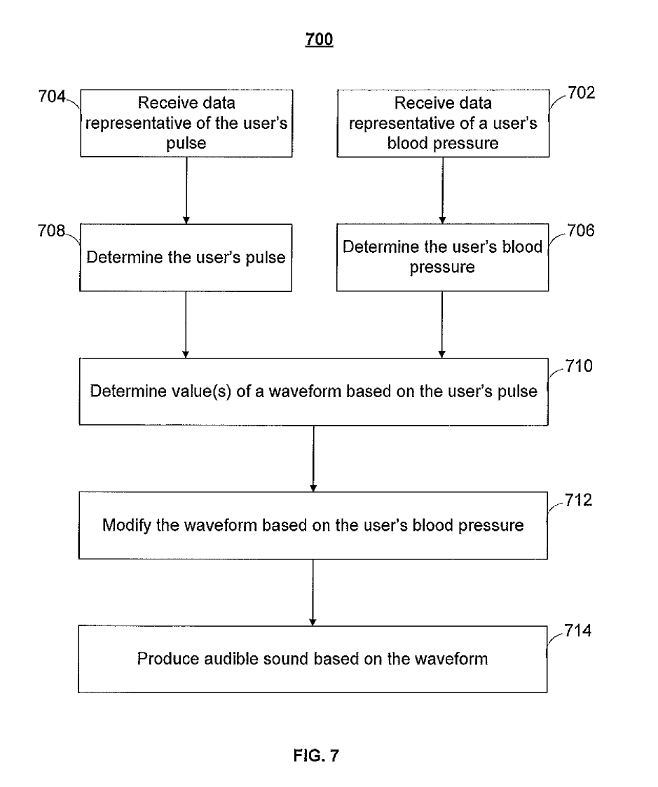

FIG. 7 shows illustrative process 700 for modifying audible indicators that are representative of a subject's pulse based on the subject's blood pressure. For example, a single beep may represent both a subject's pulse and blood pressure at the same time. At step 702, data representative of a patient's blood pressure is received. Step 702 may be substantially similar to step 602 of FIG. 6. At step 704, data representative of the patient's pulse is received. In an embodiment, the data received at steps 702 and 704 may be the same data or the data may overlap. For example, the data may have been produced and/or measured by other sensors 206 of FIG. 2. The data may be received by, for example, signal processing unit 208 of FIG. 2. At step 706, the patient's blood pressure is determined. Step 706 may be substantially similar to step 604 of FIG. 6. At step 708, the patient's pulse is determined. For example, signal processing unit 208 may determine the timing of the patient's pulse from the received pulse data received at step 704. The determination of the patient's pulse may be implemented in any suitable manner such as, for example, in the manner described above.

At step 710, values of a waveform representative of a patient's pulse are determined. For example, the waveform may represent the patient's pulse by inserting a beep when the patient's pulse occurs. In an embodiment, the patient's pulse may be represented using any suitable waveform, for example, those discussed above with regard to FIGS. 3-6.

At step 712, the waveform that represents the patient's pulse (e.g., beeps when the patient's pulse occurs) is modified to represent the patient's blood pressure as well. For example, the pitch of the beep representing pulse may be adjusted based on the value of the patient's blood pressure. For example, a pulse may be detected at two times, for example, time0 and time1. Additionally, the patient's blood pressure is measured to be 100 mmHg at time0 and 150 mmHg at time1. The waveform may represent the measured data by inserting a beep with a pitch of 100 Hz at time0 and a beep with a pitch of 150 Hz at time1. In this manner a clinician is made aware of both the patient's blood pressure and pulse with a single beep at the times when a pulse occurs. The waveform representing a pulse may be modified in any suitable manner to additionally represent blood pressure. For example, the pulse waveform may be modified according to any of the methods described above with regard to FIGS. 3-6. For example, the duration and/or volume of the pulse waveform beeps may be modified according to the patient's blood pressure. In an embodiment, the pitch, volume, and/or duration of the pulse waveform beeps may be modified according to the change in a patient's blood pressure, as discussed above with regard to process 600 of FIG. 6. As a further example, the pitch, volume, and/or duration of the pulse waveform beeps may be modified according to the patient's blood pressure deviation from a normal blood pressure, as discussed above with regard to process 500 of FIG. 5.

In an embodiment, the pulse waveform beeps would remain unaltered when the patient's blood pressure is within a range, for example, a normal range of blood pressure. For example, the pulse waveform beeps may only be based on the patient's pulse when the patient's blood pressure is between 75 mmHg to 125 mmHg. For this example, this range may represent a normal blood pressure range or a range of blood pressures designated as safe for any suitable reason. If the patient's blood pressure falls below 75 mmHg, the pulse waveform beeps may be modified to represent the drop in blood pressure, the deviation from normal blood pressure, and/or the measured value of blood pressure. Additionally, if the patient's blood pressure rises above 125 mmHg, the pulse waveform beeps may be modified to represent the rise in blood pressure, the deviation from normal blood pressure, and/or the measured value of blood pressure. In an embodiment, the modification of the pulse waveform beeps may occur until an alert is sounded. For example, if an alarm is set to be activated at 150 mmHg, the pulse waveform beeps would be modified to represent the patient's blood pressure until the patient's blood pressure exceeds 150 mmHg; at which point the alarm would be initiated.

At step 714, an audible sound based on the value of the waveform determined at steps 710 and 712 is produced. Step 714 may be substantially similar to step 612 of FIG. 6.

In practice, one or more stages shown in process 700 may be combined with other stages, performed in any suitable order, performed in parallel (e.g., simultaneously or substantially simultaneously), or removed. For example, the determination of a patient's blood pressure at step 706 and the determination of a patient's pulse at step 708 may occur substantially simultaneously. Process 700 may be implemented using any suitable combination of hardware and/or software in any suitable fashion.

It should be noted that one or more stages shown in processes 300-700 of FIGS. 3-7, respectively, may be combined with other stages in any of the processes, performed in any suitable order, performed in parallel (e.g., simultaneously or substantially simultaneously), or removed. Additionally, the waveforms discussed in any given process may be equally applied to any other process without departing from the scope of the disclosure. For example, the waveforms discussed in regard to process 700 of FIG. 7 may be equally applied to process 400 of FIG. 4. Additionally, any type of suitable blood pressure or heart rate measurement may be utilized in a process that is discussed as utilizing a particular form of blood pressure or heart rate measurement. For example, diastolic and/or systolic blood pressure values may be equally applicable in a process that is discussed herein as utilizing MAP values or pulse pressure values (e.g., systolic blood pressure minus diastolic blood pressure). Any specific values of measurements or waveforms utilized in a process are discussed for illustrative purposes. Any suitable value or waveform may be equally utilized in place of the values or waveforms discussed herein.

The above described embodiments of the present discloser are presented for purposes of illustration and not of limitation, and the present disclosure is limited only by the claims which follow.

What is claimed is:

1. A method for providing audible indicators of blood pressure of a subject, comprising:
receiving data representative of the blood pressure of the subject from a sensor;
determining the subject's blood pressure based on the received data using a processor; and
producing an audible indicator that comprises at least one audible property that is based at least in part on the determined blood pressure.

2. The method of claim 1, wherein the at least one audible property that is based at least in part on the determined blood pressure comprises at least one of a duration and a volume of the audible indicator.

3. The method of claim 1, wherein the at least one audible property that is based at least in part on the determined blood pressure comprises a depth of modulation of the audible indicator.

4. The method of claim 1, wherein the audible indicator comprises a first and a second audible indicator, and wherein the first audible indicator is representative of systolic blood pressure and the second audible indicator is representative of diastolic blood pressure.

5. The method of claim 1, wherein the audible indicator is synchronized with another measured physiological parameter of the subject.

6. The method of claim 1, further comprising:
computing a difference between a normal blood pressure and the determined blood pressure.

7. The method of claim 6, wherein producing an audible indicator comprises producing an audible indicator that comprises at least one audible property that is based at least in part on the computed difference.

8. The method of claim 7, wherein the at least one audible property that is based at least in part on the computed difference comprises a sweep that is indicative of the computed difference between the normal blood pressure and the determined blood pressure.

9. The method of claim 1, further comprising:
determining a change between the determined blood pressure and a previously determined blood pressure.

10. The method of claim 9, wherein producing an audible indicator comprises producing an audible indicator that comprises at least one audible property that is based at least in part on the determined change.

11. A system for providing audible indicators of blood pressure of a subject, comprising:
a sensor configured to receive data representative of the blood pressure of the subject;
processing circuitry configured to:
determine the subject's blood pressure based on the received data; and
generate an audible waveform that comprises at least one audible property that is based at least in part on the determined blood pressure; and
a speaker configured to:
receive the generated audible waveform; and
produce an audible indicator based on the generated audible waveform.

12. The system of claim 11, wherein the at least one audible property that is based at least in part on the determined blood pressure comprises at least one of a duration and a amplitude of the audible indicator.

13. The system of claim 11, wherein the at least one audible property that is based at least in part on the determined blood pressure comprises a depth of modulation of the audible indicator.

14. The system of claim 11, wherein the audible indicator comprises a first and a second audible indicator, and wherein the first audible indicator is representative of systolic blood pressure and the second audible indicator is representative of diastolic blood pressure.

15. The system of claim 11, wherein the audible indicator is synchronized with another measured physiological parameter of the subject.

16. The system of claim 11, wherein the processing circuitry is further configured to:
compute a difference between a normal blood pressure and the determined blood pressure.

17. The system of claim 16, wherein generating an audible waveform comprises generating an audible waveform that comprises at least one audible property that is based at least in part on the computed difference.

18. The system of claim 17, wherein the at least one audible property that is based at least in part on the computed difference comprises a sweep that is indicative of the computed difference between the normal blood pressure and the determined blood pressure.

19. The system of claim 1, wherein the processing circuitry is further configured to:
determine a change between the determined blood pressure and a previously determined blood pressure.

20. The system of claim 19, wherein generating an audible waveform comprises generating an audible waveform that comprises at least one audible property that is based at least in part on the determined change.
